(19) United States
(12) Patent Application Publication
(10) Pub. No.: US 2017/0065223 A1
(43) Pub. Date: Mar. 9, 2017

(54) SYSTEMS AND METHODS FOR FAILURE DETECTION OF ENDOVASCULAR STENTS

(71) Applicants: Mehdi Razavi, Houston, TX (US); Christopher Alexander Arevalos, Houston, TX (US); Eric Stephen Richardson, Katy, TX (US)

(72) Inventors: Mehdi Razavi, Houston, TX (US); Christopher Alexander Arevalos, Houston, TX (US); Eric Stephen Richardson, Katy, TX (US)

(21) Appl. No.: 15/255,653

(22) Filed: Sep. 2, 2016

Related U.S. Application Data
(60) Provisional application No. 62/214,388, filed on Sep. 4, 2015.

(51) Int. Cl. A61B 5/00; A61F 2/07; A61B 5/027; A61B 5/07; A61B 5/0215; A61F 2/82; A61B 5/02

(52) U.S. Cl. CPC A61B 5/4851; A61F 2/82; A61F 2/07; A61B 5/02042; A61B 5/076; A61B 5/0215; A61B 5/027; A61B 5/02014; A61F 2002/823; A61F 2002/065

(57) ABSTRACT

A system including a stent further including a plurality of conductive struts disposed within a non-conductive body. The stent is configured to exclude an aneurysm within a blood vessel of a person. In addition, the system includes a first electrode and a second electrode. Further, the system includes a controller coupled to the first electrode and second electrode. The controller is configured to measure an impedance between the first electrode and the second electrode and across tissue surrounding the stent to determine if a leak has occurred between the stent and the aneurysm.

### CROSS-REFERENCE TO RELATED APPLICATIONS

This application claims benefit of U.S. provisional patent application Ser. No. 62/214,388 filed Sep. 4, 2015, and entitled “Systems And Methods For Failure Detection of Endovascular Stents,” which is hereby incorporated herein by reference in its entirety.

### STATEMENT REGARDING FEDERALLY SPONSORED RESEARCH OR DEVELOPMENT

Not applicable.

### BACKGROUND

This disclosure generally relates to stents for treating an aneurysm. More particularly, this disclosure relates to systems and methods for the detection of failures in stents.

An aneurysm is a bulge in the body of vessel, most commonly an artery. Aneurysms typically have an increased chance of rupturing if their diameter is large or if their progression (e.g., growth rate) is rapid. The aorta is the largest blood vessel in the human body. With the exception of blood circulation to the lungs, all of the body's blood supply flows through the aorta. In order for the heart to adequately circulate blood throughout the entire circulatory system it has to generate relatively high forces and pressures which are transferred to and through the aorta. As a result, the aorta is vulnerable to formation of aneurysms, particularly in the abdominal region (i.e., below the diaphragm).

Treatment of an abdominal aortic aneurysm (AAA) may involve a surgical procedure in which the abdomen is opened and the aneurysmal region(s) of the aorta are resected and replaced with a graft. These surgical procedures, while quite effective and relatively safe, nevertheless require “opening” of the patient's abdomen, thorax, or both. As a result, post operation recovery can be long and can include associated morbidities such as perioperative heart attacks, strokes, or embolization of tissue downstream to the renal arteries or the arteries feeding the legs.

Therefore, additional techniques have been developed to correct the aneurysm without the need for open surgery (and the risks associated therewith). One such technique involves the endovascular placement of a stent graft within the aorta. Once inserted within the aorta (e.g., via the femoral artery) the graft, which is in a collapsed state, is guided upstream until it is proximate to the aneurysm. Then, the stent graft is deployed by expanding it radially outward until it engages with the inner walls of the aorta both upstream and downstream of the aneurysm, thereby excluding the aneurysm from the blood flow and internal pressures of the aorta. This technique is commonly referred to as an endovascular repair of an abdominal aortic aneurysm (or a so called EVAR).

### SUMMARY

Some embodiments disclosed herein are directed toward a system including a stent. The stent includes a plurality of conductive struts disposed within a non-conductive body, and is configured to exclude an aneurysm within a blood vessel of a person. In addition, the system includes a first electrode, a second electrode, and a controller coupled to the first electrode and second electrode. The controller is configured to measure an impedance between the first electrode and the second electrode and across tissue surrounding the stent to determine if a leak has occurred between the stent and the aneurysm.

Other embodiments disclosed herein are directed toward a system including a stent. The stent includes a plurality of conductive struts disposed within a non-conductive body, and is configured to exclude an aneurysm within a blood vessel of a person. In addition, the system includes an external device disposed outside of the person, and a stent monitoring device. The stent monitoring device includes a first electrode, a second electrode, and a controller coupled to the first electrode and second electrode. The controller is configured to measure an impedance between the first electrode and the second electrode and across tissue surrounding the stent to determine if a leak has occurred between the stent and the aneurysm. In addition, the stent monitoring device includes a communication device that is configured to wirelessly communicate with the external device.

Embodiments described herein comprise a combination of features and characteristics intended to address various shortcomings associated with certain prior devices, systems, and methods. The foregoing has outlined rather broadly the features and technical characteristics of the disclosed embodiments in order that the detailed description that follows may be better understood. The various characteristics and features described above, as well as others, will be readily apparent to those skilled in the art upon reading the following detailed description, and by referring to the accompanying drawings. It should be appreciated that the conception and the specific embodiments disclosed may be readily utilized as a basis for modifying or designing other structures for carrying out the same purposes as the disclosed embodiments. It should also be realized that such equivalent constructions do not depart from the spirit and scope of the principles disclosed herein.

### BRIEF DESCRIPTION OF THE DRAWINGS

For a detailed description of various exemplary embodiments, reference will now be made to the accompanying drawings in which:

FIG. 1 is a schematic, partial cross-sectional view of a system for detecting failures in an endovascular graft stent in accordance with at least some embodiments;

FIG. 2 is a block diagram of the system of FIG. 1 in accordance with at least some embodiments;

FIG. 3 is a method in accordance with at least some embodiments; and

FIG. 4 is a schematic, partial cross-sectional view of another system for detecting failures in an endovascular graft stent in accordance with at least some embodiments.

### DETAILED DESCRIPTION OF EXEMPLARY EMBODIMENTS

The following discussion is directed to various exemplary embodiments. However, one of ordinary skill in the art will understand that the examples disclosed herein have broad application, and that the discussion of any embodiment is meant only to be exemplary of that embodiment, and not intended to suggest that the scope of the disclosure, including the claims, is limited to that embodiment.

The drawing figures are not necessarily to scale. Certain features and components herein may be shown exaggerated in scale or in somewhat schematic form and some details of conventional elements may not be shown in interest of clarity and conciseness.

In the following discussion and in the claims, the terms “including” and “comprising” are used in an open-ended fashion, and thus should be interpreted to mean “including, but not limited to . . . .” Also, the term “couple” or “couples” is intended to mean either an indirect or direct connection. Thus, if a first device couples to a second device, that connection may be through a direct connection of the two devices, or through an indirect connection that is established via other devices, components, nodes, and connections. In addition, as used herein, the terms “axial” and “axially” generally mean along or parallel to a given axis (e.g., central axis of a body or a port), while the terms “radial” and “radially” generally mean perpendicular to the given axis. For instance, an axial distance refers to a distance measured along or parallel to the axis, and a radial distance means a distance measured perpendicular to the axis.

As previously described, one treatment for an abdominal aortic aneurysm is the endovascular insertion of a stent graft that excludes the aneurysm from the blood flow and interal pressures within the aorta (i.e., an EVAR). The major benefit to these procedures is that no surgical access or opening of the abdomen or thorax is required. However, because an EVAR does not involve the removal of the aneurysmal region(s)from the aorta (such as would be the case under some of the surgical treatments described above), there is a continued risk that blood will leak into the aneurysm sac. If the leak is severe enough the pressures of the aorta can once again be transmitted to the aneurysm sac, thereby potentially leading to a rupture of the aneurysm (and the possible death of the patient). Another potential complication that can occur with an EVAR procedure is a dissection of the aortic wall, where blood “seeps” between the layers of the aorta creating a tract therein. Dissections can lead to absence of flow in downstream regions, and therefore, an aortic dissection above the renal arteries (which feed the kidneys) can lead to blockage of the renal artery and infarction of the kidneys.

Leaks and dissections can occur up to a year, or possibly longer, after the EVAR procedure. Early detection of these complications is critical to their management and treatment. Conventionally, a patient who has undergone an EVAR must undergo regular computer tomography (CT) scans to monitor for the occurrence of a leak or dissection. Each CT scan (depending on the scanner and technique) can expose the patient to relatively high amounts of radiation (e.g., radiation equivalent to 800 chest x-rays). In addition, intravenous contrast is often required during a CT scan, which can be toxic to the kidneys. Thus, a testing procedure and system for evaluating the integrity of a graft stent installed during an EVAR procedure without the need for a CT scan (or even with a reduced need for such scans) would be highly desirable. Accordingly, embodiments disclosed herein include systems and methods for monitoring an endovascular stent graft for a leak or other failure by monitoring the impedance of the aneurysm and surrounding tissue (including the graft stent itself).

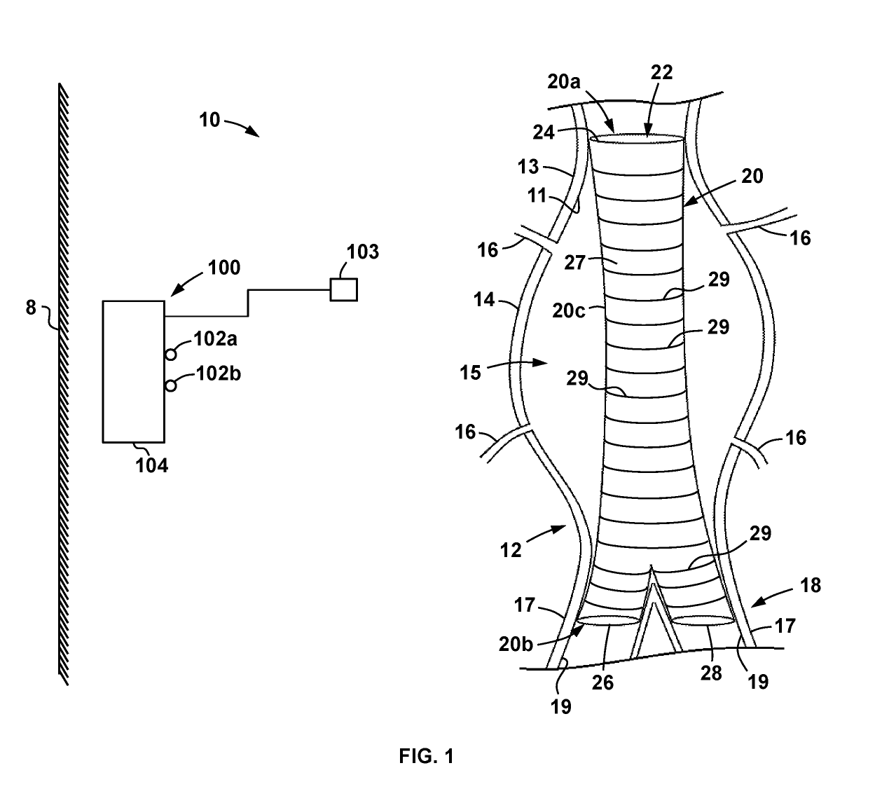

Referring now to FIG. 1, a system 10 for detecting failures in an endovascular graft stent is shown. System 10 generally includes a graft stent 20 (or more simply, a “stent 20”) and a stent monitoring device 100. Stent 20 is inserted within the aorta 12 of a person. In particular, stent 20 is inserted within the section or portion of the aorta 12 that extends through the abdominal region (e.g., below the diaphragm) of the person. In this region, the aorta 12 includes an inner wall 11, an outer wall 13, and a branch or split 18 that separates the aorta 12 into a pair of independent vessels 17 (known as the iliac vessels). As previously described above, the abdominal section of the aorta 12 is particularly vulnerable to the formation of aneurysms such as, for example, aneurysm 14 which is depicted as a radial bulge in the vessel wall of the aorta 12 upstream of the vessels 17. As aneurysm 14 grows (i.e., as the diameter of aneurysm 14 grows), the wall stresses experienced by the aorta 12 at aneurysm 14 increase. These wall stresses are further increased if the person is experiencing high blood pressure (e.g., hypertension). Eventually, if the wall stresses increase above some threshold limit, the wall of aorta 12 at aneurysm 14 may rupture thereby compromising the integrity of aorta 12 and risking the life of the person.

Stent 20 is a generally tubular member that includes a first or upper end 20a, a second or lower end 20b opposite upper end 20a, a radially outer surface 20c extending between ends 20a, 20b, and a throughbore 22 extending between ends 20a, 20b. Throughbore 22 is in communication with the inner lumen of the aorta 12 through an inlet 24 disposed at upper end 20a, and a pair of outlets 26, 28 disposed at lower end 20b. Inlet 24 is disposed within aorta 12 upstream of aneurysm 14, while each outlet 26, 28 is disposed within one of the vessels 17, downstream of aneurysm 14. In addition, the radially outer surface 20c of stent 20 engages with the inner wall 11 of aorta 12 proximate upper end 20a, and with the inner walls 19 of vessels 17 proximate lower end 20b. Thus, aneurysm 14 is excluded from the blood flowing through aorta 12 such that an excluded region or sac 15 is formed radially between radially outer surface 20c of stent 20 and the inner wall 11 of aorta 12 in the region of aneurysm 14. Therefore, following installation of stent 20, as blood flows through aorta 12 toward aneurysm 14, it is routed into inlet 24 through stent 20 and out of one of the outlets 26, 28 in vessels 17, and is prevented from communicating with sac 15. As a result, the wall tension in the aorta 12 around aneurysm 14 is reduced thereby also reducing the risk of a rupture.

Referring still to FIG. 1, stent 20 is generally constructed from a body 27 made of a fabric and a plurality of struts 29 that provide structural support to body 27. Struts 29 preferably comprise an electrically conductive material, such as, for example a metal material (e.g., nitinol, stainless steel, etc.). In addition, the fabric making up body 27 preferably comprises an electrically non-conductive material. As a result, in at least some embodiments, the struts 29 are electrically insulated from one another along stent 20 by the fabric making up body 27.

In this embodiment, stent monitoring device 100 is inserted under the skin 8 of the person proximate aorta 12. Stent monitoring device 100 includes a main body or casing 104, and a plurality of electrical contacts or electrodes, which in this embodiment comprises two electrodes 102a, 102b, although more than two electrodes are possible in other embodiments. In particular, in some embodiments, stent monitoring device 100 is inserted under the skin 8 of the person on the posterior side of the torso (i.e., along the back or rear flanks of the torso). However, other insertion locations are possible in other embodiments. Further, it should also be appreciated that stent monitoring device 100 may be disposed on the skin 8 outside the body of the person in other embodiments.

Stent monitoring device 100 is configured and operable to sense or monitor for a failure in exclusionary performance of stent 20 about aneurysm 14. Specifically, as previously described above, even though the installation of stent 20 in aorta 12 is designed to prevent fluid and pressure communication to the sac 15 of aneurysm 14, leaks and other complications (e.g., dissections) can still occur which allow blood flowing through aorta 12 to leak into sac 15, thereby increasing the risk of a rupture of aneurysm 14. For example, blood may leak between the radially outer surface 20c of stent 20 and inner wall 11 of aorta 12 at or proximate to upper end 20a or between radially outer surface 20c and the inner walls 19 of vessels 17 (which is commonly referred to as an “endoleak”). As another example, blood may fill sac 15 through one or more collateral blood vessels 16 disposed on an around outer wall 13 of aorta 12. Further, blood may migrate through the aortic wall itself between inner wall 11 and outer wall 13 in the region around aneurysm 14 (a process referred to as dissection). For convenience, blood flow along any one or more of these possible leak paths is referred to herein as a bleed complication.

Thus, stent monitoring device 100 is configured to measure an electrical impedance of the aneurysm 14 and the tissue surrounding aneurysm 14 to determine if a bleed complication has occurred or is occurring. Specifically, stent monitoring device 100 may inject a known electric current through one of the electrodes 102a, 102b which is conducted through the soft tissue of the person and into and around the electrically conductive struts 29 of stent 20. The electrical current is then received or sensed by the other of the electrodes 102a, 102b such that a voltage differential forms between the electrodes 102a, 102b.

As will be described in more detail below, a controller within stent monitoring device 100 (or a controller within some other device, which may be disposed outside the person's body) calculates the resulting impedance from known current and measured voltage to determine whether a bleed complication has occurred (e.g., if blood is flowing into sac 15 or if a dissection has occurred in aneurysm 14). Alternatively, stent monitoring device 100 may induce a known voltage across electrodes 102a, 102b and measure or sense the resulting current to calculate impedance and determine whether a bleed complication has occurred.

Because struts 29 are electrically insulated from one another along the body 27 of stent 20 as previously described, when the electric current emitted from electrodes 102a, 102b (or the electric current resulting from the voltage differential placed across electrodes 102a, 102b) conducts through struts 29, it is further conducted between adjacent struts 29 through the surrounding tissue (i.e., the aorta 12, aneurysm 14, etc.). Thus, the impedance calculated by stent monitoring device 100 represents the impedance of the tissue disposed between electrodes 102a, 102b and stent 20, as well as the tissues and fluids surrounding stent 20.

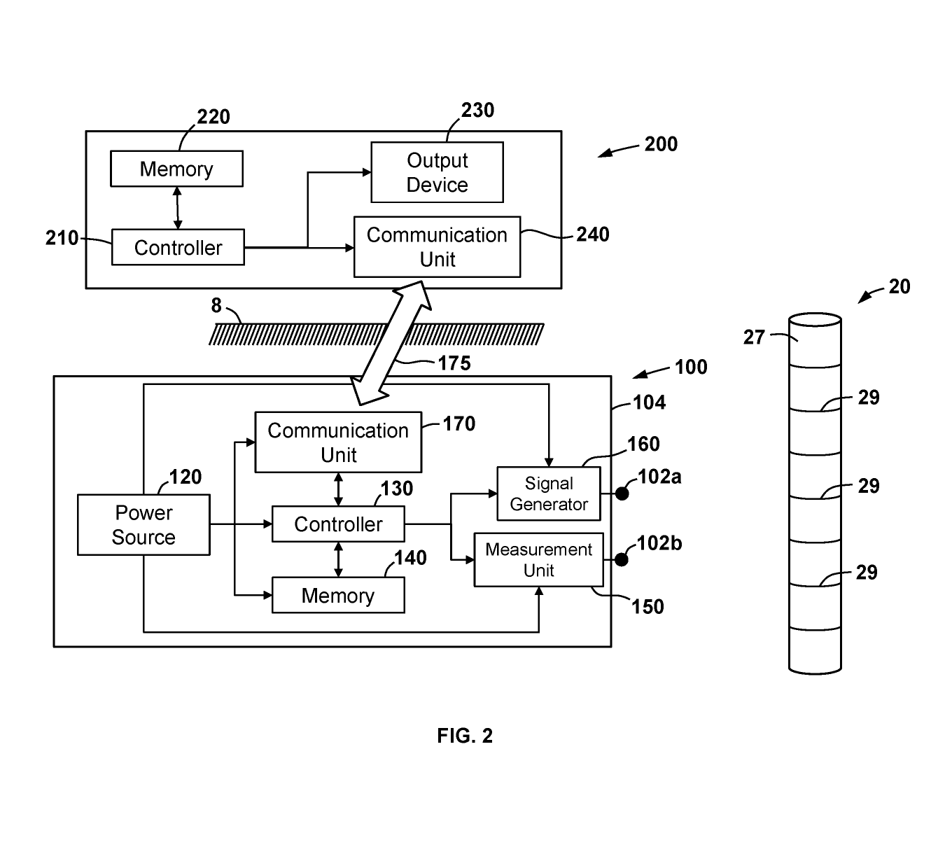

Referring now to FIG. 2, a block diagram of system 10, including stent monitoring device 100 is shown. As shown in the example of FIG. 2, the stent monitoring device 100 includes a power source 120, a controller 130, a memory 140, a communication unit 170, a measurement unit 150, and a signal generator 160. The components of stent monitoring device 100 (e.g., power source 120, controller 130, memory 140, communication unit 170, measurement unit 150, signal generator 160, etc.) may each include an electrical circuit or collection of electrical circuits configured and operable to carry out the functions discussed herein.

The power source 120 may comprise a battery (disposable or rechargeable), a charged capacitor, a wireless power receiver (e.g., inductive coil, etc.), or other sources of electrical power. The power source 120 provides electrical power to the other components within stent monitoring device 100 (e.g., controller 130, communication device 170, memory 140, measurement unit 150, signal generator 160, etc.).

The controller 130 executes software provided on memory 140, and upon executing the software on memory 140 provides the stent monitoring device 100 with all of the functionality described herein. Specifically, in some embodiments, controller 130 may comprise a hardware processor, microcontroller embedded controller, microprocessor, ASIC (application specific integrated circuit), and/or any other type of circuit that can perform the functions discussed herein.

The memory 140 may comprise volatile storage (e.g., random access memory), non-volatile storage (e.g., flash storage, read only memory, etc.), or combinations of both volatile and non-volatile storage. Data consumed or produced by the software can also be stored on memory 140. For example, measured current or voltage values, computed impedance values, etc. can be stored on memory 140 pending transmission (wireless or wired) to an external apparatus (e.g., external apparatus 200 discussed below).

The communication device 170 may be implemented in accordance with any suitable wireless protocol (e.g., BLUETOOTH®, WiFi, near field communications, radio-frequency communications, etc.) or wired communication system (e.g., an electrical conductor, fiber optic cable, etc.). In this embodiment, communication device 170 is a wireless communication device that is configured to communicate with an external apparatus 200, that is disposed outside the body of the person, through a wireless signal path 175 that is directed across the skin 8 of the person. The communication device 170 may be capable of transmitting only, or may be capable of transmitting and receiving. The controller 130 causes the communication device 170 to transmit values indicative of impedance (current, voltage) or impedance values themselves to external apparatus 200 via wireless signal path 175. The communication device 170 may be a bi-directional device to permit outgoing transmissions of data, as well as receive incoming commands from an external apparatus (e.g., external apparatus 200). For example, an external apparatus may send a command to the controller 130 via the communication device 170 to command the stent monitoring device 100 to initiate a process by which impedance is determined, or to transmit previously stored data (e.g., current, voltage, and/or impedance).

The signal generator 160 receives power from the power source 120 and generates a current or voltage under control by the controller 130. The signal generator 160 may generate a predetermined current or voltage, and is broadly referred to as a signal generator to indicate either or both possibilities. The signal generator 160 is connected to electrodes 102a. As a current source, the signal generator 160 injects a current through electrode 102a.

The measurement unit 150 measures the resulting voltage or current. That is, if the signal generator 160 injects a predetermined current into the person via electrode 102a, the measurement unit 150 measures the resulting voltage across electrodes 102a, 102b. If the signal generator 160 imposes a predetermined voltage across electrodes 102a, 102b, the measurement unit 150 measures the resulting current (e.g., using a sense resistor). In either case, the measurement unit 150 provides the measured electrical parameter to the controller 130.

The controller 130 thus knows the magnitude of the predetermined current or voltage generated by the signal generator 160 and the magnitude of the measured voltage or current from the measurement unit 150. As such, the controller 130 can compute impedance (and does so as the ratio of voltage to current), and transmit the computed impedance to the external apparatus 200 via communication device 170. However, as noted above, the controller 130 may not compute impedance and instead transmit the measured electrical parameter (voltage or current) to the external apparatus 200 for the external apparatus 200 to compute impedance. The external apparatus 200 may or may not know what predetermined current or voltage was set by the signal generator 160. If the external apparatus 200 does know the magnitude of the current/voltage generated by the signal generator 160, that value need not be (but of course can be) transmitted to the external apparatus 200 by controller 130. If the external apparatus is not aware of the magnitude of the current/voltage generated by the signal generator 160, the controller 130 preferably transmits both the measured voltage/current from the measurement unit 150 and the signal generator 160's predetermined current/voltage. In addition, it should be appreciated that impedance may be determined at a frequency of 0 Hz (i.e., DC), or at one or more frequencies (i.e., AC).

Referring still to FIG. 2, external apparatus 200 includes a controller 210, a communication device 240, a memory 220, and at least one output device 230. The external apparatus 200 may be computer (desktop, laptop, notebook, etc.), a smart phone, or any other type of device capable of interacting with the stent monitoring device 100 either wirelessly or through a wired connection. In some embodiments, the external apparatus 200 is, or is built into, a bedside monitor. The components of external apparatus 200 (e.g., controller 210, communication device 240, memory 220, output device(s) 230, etc.) may each include an electrical circuit or collection of electrical circuits configured and operable to carry out the functions discussed herein.

The controller 210 executes software provided on memory 220, and upon executing the software on memory 220 provides the external apparatus 200 with all of the functionality described herein. Specifically, in some embodiments, controller 210 may comprise a hardware processor, microcontroller embedded controller, microprocessor, ASIC (application specific integrated circuit), and/or any other type of circuit that can perform the functions discussed herein.

Memory 220 may be the same or similar to memory 140 in stent monitoring device 100 and may store software for execution by controller 210, as well as data (including impedance, current, voltage, etc. from stent monitoring device 100).

Communication device 240 may be the same or similar to the communication device 170 of stent monitoring device 100. In this embodiment, communication device 240 receives either the computed impedance values or data indicative of impedance (e.g., current, voltage, etc.) via wireless signal 175 (which may instead comprise a wired communication path as explained above). If external device 200 receives only the raw measured current and/or voltage from stent monitoring device 100, controller 210 may further compute the impedance in the same manner as discussed above.

Regardless of whether the external apparatus 200 receives the computed impedance values from stent monitoring device 100 or computes the impedance values from the measured values (current, voltage) from stent monitoring device 100, controller 210 in external apparatus 200 may send the impedance values (or some information indicative of the impedance values) to one or more of the output devices 230. Output devices 230 may comprise any suitable device or apparatus for displaying or communicating data or determinations by system 10. For example, output device 230 may comprise display (e.g., a LCD display, plasma display, etc.) that shows the computed impedance values and/or other information and parameters, such that a physician may ultimately determine whether a bleed complication has occurred in aneurysm 14.

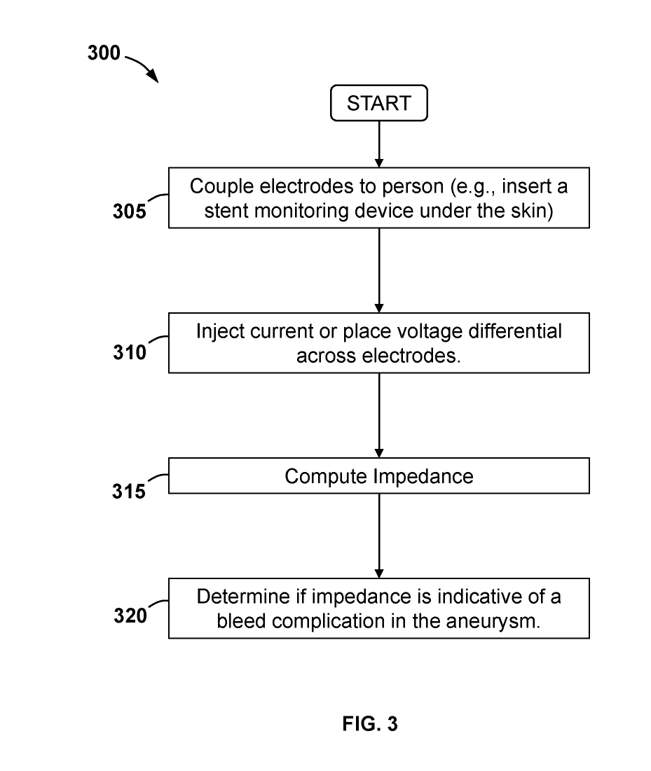

FIG. 3 shows an illustrative method 300. At 305, one or more electrodes are coupled to a person. In the embodiment of FIGS. 1 and 2, one or more electrodes (e.g., electrodes 102a, 102b) are disposed on body 104 of stent monitoring device 100 and are coupled to the person by inserting the stent monitoring device 100 under the skin 8 of the person. At 310, upon a user activating a control, either (1) a current is injected into the body in a region proximate an aneurysm and endovascular stent graft via one or more of the electrodes; or (2) a voltage differential is placed across the electrodes to induce a current that conducts through the aneurysm, stent, and surrounding tissue. In the embodiment of FIGS. 1 and 2, a user may cause controller 130 in stent monitoring device 100 to inject a current or place a voltage differential across two or more of the electrodes (e.g., electrodes 102a, 102b) by activating a control (e.g., pushing a button) on external apparatus 200 or some other device (which then commands controller 130 to inject the current or apply a voltage via the signal generator 160 as discussed above). At 315, the method 300 includes computing the impedance from the ratio of the injected current to resulting voltage or the applied voltage and the resulting current from 310. For example, in the embodiment of FIGS. 1 and 2, the impedance may be calculated by the controller 130 or controller 210 by computing the ratio of the injected current and measured voltage at electrodes 102a, 102b or by computing the ratio of the applied voltage and resulting current at electrodes 102a, 102b.

At 320, the method 300 includes determining if the impedance computed at 315 is indicative of a bleed complication in the aneurysm. Since blood generally has a lower impedance than other materials and tissues within the body (e.g., bone, fat tissue, air, etc.) the accumulation of blood in the measured area (i.e., around the stent 20 and aneurysm 14) should cause the impedance to drop or decrease over time. Thus, at 320, one may monitor for this drop or decrease to determine that a bleed complication has likely occurred. For example, it may be determined that the impedance computed at 315 is below some threshold, which may be an established baseline, thereby indicating that additional blood has entered into the aneurysm sac (e.g., sac 15 in FIG. 1) or has dissected through the wall of the aorta (e.g., between walls 11, 13 of aorta 12). The threshold value may be computed based on initial conditions upon initially coupling the electrodes to the person (e.g., just after inserting the stent monitoring device 100 under skin 8). Specifically, in the embodiment of FIGS. 1 and 2, upon initially inserting stent monitoring device 100 under the skin 8 of the person, an initial current and voltage reading is taken (or a plurality of initial current and voltage readings are taken) to establish an initial impedance (or average impedance) or the stent 20, aneurysm 14, and surrounding tissues and fluids. This initial impedance (or initial average impedance) is then utilized to establish a baseline or threshold for comparison to all subsequent impedance measurements. Thereafter, after each impedance measurement by system 10, the controller 130 or the controller 210 may determine whether the currently measured impedance is sufficiently different from the threshold value or baseline to indicate that a potential bleed complication has occurred. As a result, if the measured impedance is sufficiently different than the threshold (e.g., the measured impedance is outside some predetermined range based on the threshold), system 10 (e.g., via external device 200) may then alert the user (e.g., a physician) to the occurrence of a bleed complication in aneurysm 14.

Referring back now to FIG. 1, in some embodiments, the stent monitoring device 100 is configured to take measurements of current and/or voltage at times when a bleed complication (e.g., leak, dissection, etc.) is more likely to occur. In particular, the heart repetitively contracts to pump blood flow through the circulatory system (including the aorta). A contraction of the heart results in an increase in pressure within the aorta (and other blood vessels), while a relaxation of the heart results in a decrease in pressure. The period of time associated with the increase in pressure within the circulatory system due to a contraction of the heart is known as systole, and the period of time associate with the decrease in pressure within the circulator system due to a relaxation of the heart is known as diastole. Because the pressure within a blood vessel is highest during systole, a leak or dissection of blood at aneurysm 14 is much more likely to occur during this period.

Therefore, in some embodiments, an additional force sensor 103 may be disposed on or simply coupled to stent monitoring device 100 to sense or measure the increase in pressure and allow stent monitoring device 100 (via controller 130) to measure the current or voltage of the tissue surrounding aneurysm 14 and stent 20 to allow for computation of the impedance during this relatively high pressure period. In some of these embodiments, the force sensor may measure the increase in pressure by detecting the pressure increase within the aorta 12 itself or by sensing the pressure pulsation emitted by the aorta 12 within the surrounding tissue during systole (e.g., such as in the embodiment shown in FIG. 1). Although not specifically shown, force sensor 103 may be coupled to controller 130.

In other embodiments, stent monitoring device 100 is configured to sense the electrical signals associated with the contraction of the heart during systole and time the measurement of current and/or voltage to match the systolic period. The sensing of the electrical signals associated with the contraction of the heart may be accomplished by sensing through one or more of the electrodes 102a, 102b or through an additional electrical contact disposed along or in body 104 of stent monitoring device 100.

By timing the measurement of current and/or voltage with the systolic and/or diastolic periods, system 10 may be able to determine the impedance change within the aneurysm 14, stent 20, and surrounding tissues and fluids over time and as a function of the pressure cycles within aorta 12 (i.e., during systole and diastole). Without being limited to this or any other theory, the ability to track impedance changes as a function of the systolic and/or diastolic periods allows system 10 (e.g., via controllers 130, 210) to filter the determined impedances to avoid a false indication of a bleed complication when the impedance changes (e.g., decreases) as a result of some other type of physiological event (i.e., other than a bleed complication). For example, a buildup of fluid in an adjacent organ (e.g., lungs, bladder, etc.) may cause the impedance detected by system 10 to drop over time, but the decreases in impedance will not necessarily be aligned or synced with the systolic or diastolic periods (e.g., because the heart is not pumping the fluids in question). By contrast, a bleed complication for stent 20 and aneurysm 14 should manifest both as a change in impedance over a period of time (e.g., due to the progressively increasing volume of blood either within the aneurysm sac 15 and/or within the walls of aorta 12 at aneurysm 14) and as a plurality of individual changes in impedance that occur during the systolic periods (e.g., because the pressure is highest within aorta 12 during this period such that the above referenced bleed complications are more likely to occur).

Therefore, in some embodiments, system 10 may differentiate between an impedance change that is associated with a bleed complication in an aneurysm (e.g., aneurysm 14) and an impedance change that is associated with some other physiological event (e.g., other non-blood fluid accumulations). Specifically, in some embodiments, system 10 may detect impedance at least once during each systolic period and at least once during each diastolic period, and may then determine if a change (e.g., a decrease) in impedance noted over a period of time is likely due to a bleed complication or some other physiological event (e.g., build up of fluid in lungs and/or bladder) by determining whether the impedance changes are occurring only or mostly during the systolic period or whether the changes are occurring uniformly during both the systolic and diastolic periods. A series of impedance changes that occur only or mostly during the systolic periods and not during the diastolic periods would tend to indicate a bleed complication, because these fluid accumulations are driven by the pressure generated by the heart (which is greatest during systole). By contrast, a series of impedance changes that do not significantly track or align with the systolic or diastolic period (e.g., a series of impedance changes that seem to occur uniformly during both systole or diastole) would tend to indicate a fluid accumulation due to some other physiological event unrelated to a bleed complication of the stent 20 and aneurysm 14.

Alternatively, in some embodiments, system 10 may determine impedance at the beginning and end of each systolic period and then determine whether the changes in impedance occurring over time are only or mostly occurring during the systole. For example, system 10 may determine that the impedance changes occur only or mostly during the period between the beginning and end of each systolic period by determining that no change or an insignificant change in impedance occurs between the end of each systolic period and the beginning of the next successive systolic period (e.g., following the intervening diastolic period). However, it should be noted that in other embodiments, other specific methods and logic may be used to determine that the changes in impedance (e.g., decreases) are substantially synced with the systolic period that are not specifically described herein.

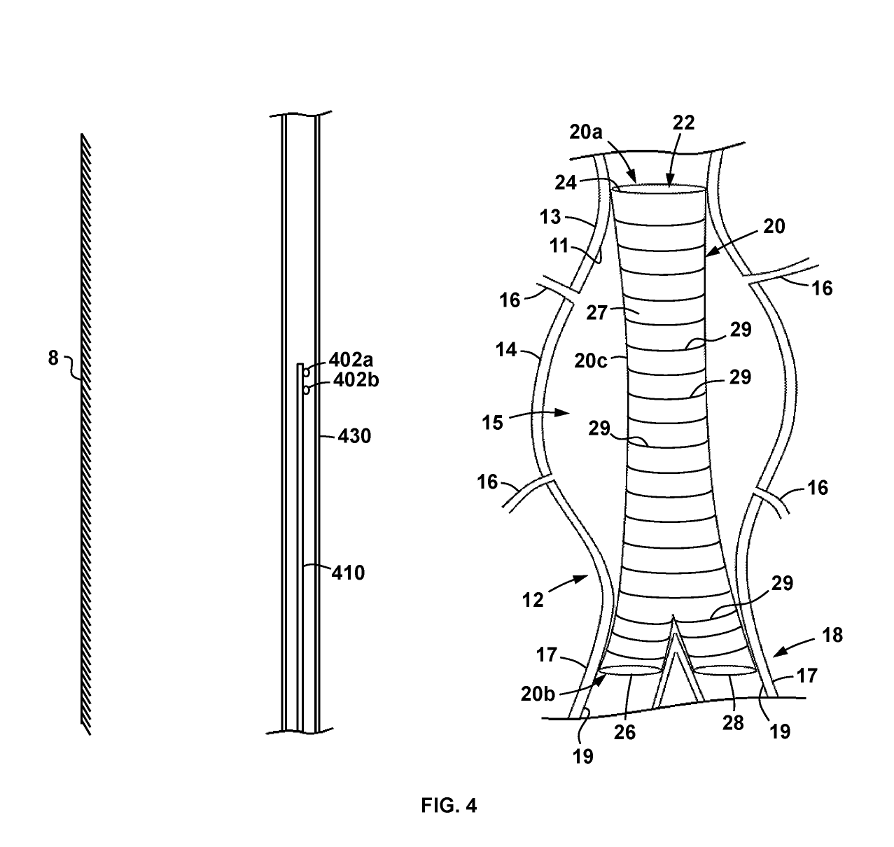

While embodiments disclosed herein have included a stent monitoring device 100 that is disposed under the skin 8 of a person, it should be appreciated that other devices may be used to measure the impedance of the aortic aneurysm 14 and the surrounding tissue. For example, referring now to FIG. 4, where a system 400 for detecting failures of a graft stent inserted within an aorta (e.g., leaks, dissections, etc.) is shown. System 400 generally includes the previously described stent 20; however, system 400 does not include stent monitoring device 100 (see FIG. 1). Rather, system 400 includes a catheter 410 that is inserted within another blood vessel 430 that runs proximate to aorta 12. In particular, in some embodiments, catheter 410 is inserted within the inferior vena cava. Catheter 410 includes a plurality of electrodes, which in this case includes two electrodes 402a, 402b. Electrodes 402a, 402b are coupled to other electronic devices similar to those discussed above for stent monitoring device 100 (e.g., controller 130, memory 140, signal generator 160, measurement unit 150, etc.) through an electrical conductor (not shown) that is routed along and/or through catheter 410 or through a wireless connection. During use, electrical current is injected into the tissue surrounding aneurysm 14 and stent 20 via electrodes 402a, 402b or a voltage differential is applied across electrodes 402a, 402b to determine the impedance of the aneurysm 14 and surrounding tissue in the same manner as described above for system 10. Thus, a detailed description of the operation and use of system 400 is omitted in the interests of brevity.

In the manner described, by monitoring the impedance of the tissue surrounding an endovascular stent (e.g., stent 20) inserted to exclude an aneurysm (e.g., aneurysm 14), a bleed complication (e.g., a leak into the aneurysm sac or dissection through the vascular wall) may be detected without performing a CT scan. Therefore, performance of a CT scan can be limited to characterizing the exact location and nature of the bleed complication after initial detection thereof (rather than use of a CT scan to determine the existence of a bleed complication in the first place). As a result, the number of CT scans that a person must undergo for detection and treatment of a bleed complication resulting from an EVAR procedure is greatly reduced.

While exemplary embodiments have been shown and described, modifications thereof can be made by one skilled in the art without departing from the scope or teachings herein. The embodiments described herein are exemplary only and are not limiting. Many variations and modifications of the systems, apparatus, and processes described herein are possible and are within the scope of the protection. For example, while the systems and methods described herein have been discussed for detecting a bleed complication associated with an abdominal aortic aneurysm, it should be appreciated that the systems and methods described herein may be employed with stents inserted to exclude aneurysms in other types of blood vessels within the body (e.g., other arteries or veins within the body).

Accordingly, the scope of protection is not limited to the embodiments described herein, but is only limited by the claims that follow, the scope of which shall include all equivalents of the subject matter of the claims. Unless expressly stated otherwise, the steps in a method claim may be performed in any order. The recitation of identifiers such as (a), (b), (c) or (1), (2), (3) before steps in a method claim are not intended to and do not specify a particular order to the steps, but rather are used to simplify subsequent reference to such steps.

What is claimed is:

1. A system, comprising:
a stent including a plurality of conductive struts disposed within a non-conductive body, wherein the stent is configured to exclude an aneurysm within a blood vessel of a person;
a first electrode;
a second electrode; and
a controller coupled to the first electrode and second electrode, the controller configured to measure an impedance between the first electrode and the second electrode and across tissue surrounding the stent to determine if a leak has occurred between the stent and the aneurysm.

2. The system of claim 1, wherein the first electrode and the second electrode are both coupled to a stent monitoring device that is configured to be inserted under the skin of the person.

3. The system of claim 2, wherein the stent monitoring device includes a communication device that is configured to wirelessly communicate with an external device disposed outside of the person.

4. The system of claim 1, wherein the first electrode and the second electrode are disposed on a catheter that is configured to be inserted within a blood vessel of the person.

5. The system of claim 1, further comprising:
a signal generator coupled to the first electrode; and
a measurement unit coupled to the second electrode;
wherein the controller is configured to:
inject a known current into the person and one or more of the conductive struts, through the first electrode with a signal generator;
measure a resulting voltage at the second electrode with a measurement unit; and
compute the impedance of the tissue surrounding the stent as a ratio of the known current and the resulting voltage.

6. The system of claim 5, wherein the controller is further configured to compare the computed impedance to a predetermined threshold value.

7. The system of claim 1, wherein the controller is configured to:
measure the impedance at least once during each systolic pressure increase within the blood vessel;
measure the impedance at least once during each diastolic pressure decrease within the blood vessel; and
differentiate a change in impedance over time caused by a leak between the stent and the aneurysm from a change in impedance over time due that is not due to a leak between the stent and the aneurysm.

8. The system of claim 1, wherein the controller is configured to measure the impedance during each systolic pressure increase within the blood vessel.

9. The system of claim 8, wherein the controller is configured to sense an electrical signal associated with a contraction of the heart of the person via at least one of the first electrode and the second electrode.

10. The system of claim 8, further comprising a force sensor coupled to the controller, wherein the force sensor is configured to sense an increase in pressure within the blood vessel of the person.

11. A system, comprising:
a stent including a plurality of conductive struts disposed within a non-conductive body, wherein the stent is configured to exclude an aneurysm within a blood vessel of a person;
an external device disposed outside of the person; and
a stent monitoring device further comprising:
a first electrode;
a second electrode;
a controller coupled to the first electrode and second electrode, the controller configured to measure an impedance between the first electrode and the second electrode and across tissue surrounding the stent to determine if a leak has occurred between the stent and the aneurysm; and
a communication device that is configured to wirelessly communicate with the external device.

12. The system of claim 11, wherein the stent monitoring device is configured to be inserted under the skin of the person.

13. The system of claim 11, wherein the stent monitoring device further comprises:
a signal generator coupled to the first electrode; and
a measurement unit coupled to the second electrode;
wherein the controller is configured to:
inject a known current into the person and one or more of the conductive struts, through the first electrode with a signal generator;
measure a resulting voltage at the second electrode with a measurement unit; and
compute the impedance of the tissue surrounding the stent as a ratio of the known current and the resulting voltage.

14. The system of claim 13, wherein the controller is further configured to compare the computed impedance to a predetermined threshold value.

15. The system of claim 11, wherein the controller is configured to:
measure the impedance at least once during each systolic pressure increase within the blood vessel;
measure the impedance at least once during each diastolic pressure decrease within the blood vessel; and
differentiate a change in impedance over time caused by a leak between the stent and the aneurysm from a change in impedance over time due that is not due to a leak between the stent and the aneurysm.

16. The system of claim 11, wherein the controller is configured to measure the impedance during each systolic pressure increase within the blood vessel.

17. The system of claim 16, wherein the controller is configured to sense an electrical signal associated with a contraction of the heart of the person via at least one of the first electrode and the second electrode.

18. The system of claim 16, further comprising a force sensor coupled to the controller, wherein the force sensor is configured to sense an increase in pressure within the blood vessel of the person.
